(19) United States
(12) Patent Application Publication
(10) Pub. No.: US 2023/0330445 A1
(43) Pub. Date: Oct. 19, 2023

(54) DENTAL WHITENING COMPOSITION

(71) Applicants: Sam Alam, Houston, TX (US); Ryan Alam, Houston, TX (US)

(72) Inventors: Sam Alam, Houston, TX (US); Ryan Alam, Houston, TX (US)

(21) Appl. No.: 17/720,164

(22) Filed: Apr. 13, 2022

(51) Int. Cl. A61Q 11/00; A61K 8/02; A61K 8/66; A61K 8/92; A61K 8/21

(52) U.S. Cl. CPC A61Q 11/00; A61K 8/0208; A61K 8/66; A61K 8/922; A61K 8/21; A61K 2800/28; A61K 2800/48

(57) ABSTRACT

A dental whitening composition includes an effective amount of at least one fruit enzyme, an effective amount of a fruit sweetener, and an effective amount of abrasive material. The at least one fruit enzyme and the at least one fruit sweetener are in an aqueous suspension with the abrasive material. Dental whitening wipes have the foregoing composition disposed in an absorbent material for temporary application to teeth.

### CROSS REFERENCE TO RELATED APPLICATIONS

Not Applicable.

### STATEMENT REGARDING FEDERALLY SPONSORED RESEARCH OR DEVELOPMENT

Not Applicable

### NAMES OF THE PARTIES TO A JOINT RESEARCH AGREEMENT

Not Applicable.

### BACKGROUND

This disclosure relates to the field of dental whitening materials and methods.

More particularly, the disclosure relates to compositions that may be applied to human teeth using absorbent wipes to whiten teeth.

Exogenous dental staining is caused by, among other things, the existence of various bacteria on the tooth surface. Such bacteria secrete a plurality of viscous substances onto tooth surfaces. Thus, food residues and soft dirt in the daily diet, smoke stains, certain mineral substances in drinking water and mineral substances in human saliva are adsorbed in the viscous substances to form bacterial plaques and dental calculus that gradually yellow the teeth.

Dental whitening known in the art includes professionally applied teeth whitening products using certain concentration of the teeth whitening chemicals for use by professional dental practitioner. The same teeth whitening chemicals in different concentrations were later introduced for use by the public at home using, e.g., plastic strips that fit onto the teeth and mouth trays with the whitening chemicals in gel form.

Dental whitening has gained popularity with the public, which has come to demand whiter, more perfect smiles that give them a positive effect on both physical appearance and psychological health. Dental whitening is known to boosts self-confidence. The cosmetic nature of modern society has resulted in whiter teeth being part of a person's expected hygiene, thereby raising self-awareness of discolored teeth.

Dental whitening has become one of the most frequently requested dental products and procedures by the public, with access to many different types of whitening products ranging from in-dentist-office based products that use highly concentrated whitening agents to home-based products, such as whitening toothpastes, whitening gels used on mouth trays and sticky teeth whitening strips.

Teeth whitening known in the art uses two common chemicals to serve as whitening agents: hydrogen peroxide and carbamide peroxide. Highly concentrated hydrogen peroxide or carbamide peroxide compositions are applied to teeth for 60-90 minutes under the supervision of dental professionals, whereas, the lower concentration hydrogen peroxide or carbamide peroxide are sold for use by the individual using a whitening tray or stripe applied to the teeth surfaces.

The effects of known whitening compositions when applied directly on the surfaces of the teeth include eroding tooth enamel and causing gum irritation, particularly if the whitening gel and strips come in contact with the user's gums for an extended period of time. Many users are also affected by tooth sensitivity from these whitening products. Studies show that the use of hydrogen peroxide even in low concentration at 3% with 97% water can inflame the teeth roots in the gums and can damage the protective enamel of teeth. As the peroxide product bleaches away the stains on teeth, it also causes the demineralization which makes the teeth more porous. This is because, the use of peroxide cause significant damage to tooth enamel.

### SUMMARY

One aspect of the present disclosure is a dental whitening composition. A composition according to the present disclosure includes an effective amount of at least one fruit enzyme, one fruit sweetener, and at least one abrasive material. The at least one fruit enzyme, and the at least one fruit sweetener are in an aqueous suspension with the abrasive material. Dental whitening wipes in another aspect of this disclosure have the foregoing composition disposed in an absorbent material for temporary application to teeth.

In some embodiments, the at least one fruit enzyme comprises one or more of papain, malic acid or bromelain.

In some embodiments, the abrasive materials comprise charcoal and/or sodium bicarbonate.

Some embodiments further comprise an effective amount of peppermint oil.

Some embodiments further comprise an effective amount of sodium fluoride.

Some embodiments further comprise an effective amount of glycerin.

In some embodiments, the at least one fruit enzyme comprises papain in a range of 5 to 14 percent by weight, malic acid in a range of 2 to 5 percent by weight, and bromelain in a range of 4 to 11 percent by weight; the fruit sweetener comprises xylitol is in a range of 10 to 24 percent by weight; and a remainder of the mixture comprises water.

Some embodiments further comprise peppermint oil in a range of 0.5 to 1.5 percent by weight, and wherein the water is reduced in amount correspondingly to an amount of the peppermint oil.

Some embodiments further comprise glycerin in a range of 20 to 24 percent, and wherein the water is reduced in amount corresponding to an amount of the glycerin.

Other aspects and possible advantages will be apparent from the description and claims that follow.

### DETAILED DESCRIPTION

A composition and whitening strips or wipes according to the present disclosure comprise fruit enzymes work differently than known common peroxide agents. Compositions according to the present disclosure may actually help lessen the deterioration of oral health by killing tooth decay-causing bacteria that attack the tooth's surface, or enamel.

Dental plaque is one of the chief causal agents responsible for two of the most common diseases implicated in the deterioration of oral health, i.e., gingivitis and dental caries (tooth decay). Plaque is the soft, tenacious material found on tooth surfaces which is not readily removed by rinsing with water. Tooth decay is damage to a tooth that can happen when decay-causing bacteria attack the tooth's surface, or enamel, which causes enamel to lose minerals, which if not treated, can cause pain, infection and abscess, leading to increase the discoloration of teeth.

As used throughout the present disclosure, all numerical values of material fractional amounts in a composition are within a described range. An effective amount of any of the substances described herein means an amount in a composition that provides the properties described with reference to each of the foregoing substances, while being safe to use in the mount. The tooth whitening compositions disclosed herein can be in the form of a liquid (aqueous solution) or a semi-solid substance such as a gel absorbed into a medium for application to the teeth. In various embodiments described herein the following general descriptions may apply.

A whitening agent may comprise one or more fruit enzyme-related compounds, comprising distinct properties to prevent the yellowing of teeth caused by gum inflammation, tooth decay, and enamel degradation.

In some embodiments, the tooth whitening compositions disclosed herein may comprise at least 4 fruit enzymes, one of which is acidic (e.g., derived from strawberries), two may be non-acidic (e.g., derived from papaya, pineapple) and may be one low-acidic (e.g., derived from plums) which comprise distinct properties to whiten teeth. For example, papain, the main ingredient of Papacarie, is used to treat tooth decay and is reported to have healing properties that reduce inflammation. It is also gentle to dental enamel because of its non-acidic property and thus strengthens the resin on enamel. Malic acid which increases saliva production, protects against tooth decay, a common cause of discoloration, by washing away food and debris. Bromelain has shown to degrade stains on the tooth enamel surface allowing light to reflect off of the enamel surface giving the appearance of whiter teeth visible to the naked eye.

In some embodiments the tooth whitening compositions disclosed herein, materials may comprise substances extracted from two fruits: plum and pumpkins, with properties that affect dental caries. While other sugars break down into food for bacteria that spread in the mouth and cause plaque, caries, and gum disease, xylitol is indigestible by common oral bacteria. As a result, the presence of harmful bacteria in the mouth is greatly reduced. It has been shown that the use of xylitol in some circumstances restore tooth enamel by producing more alkaline saliva that enables remineralization of the teeth.

In some embodiments of the tooth whitening compositions disclosed herein, glycerin may be added to expedite stripping of moisture in the tooth enamel. This moisture stripping allows for the whitening product to reach the stains below the surface of the tooth. Furthermore, glycerin may be used as thickening agent in teeth whitening products to prolong their shelf-life.

In some embodiments, the tooth whitening compositions disclosed herein may comprise sodium bicarbonate. Sodium bicarbonate, commonly known as baking soda is salty and alkaline. Sodium bicarbonate is effective at lightening extrinsic stains caused by acidic foods, and drinks such as coffee, red wine, and tea. Baking soda also serves as an abrasive, making it beneficial for removing plaque and stain build-up. The anti-caries and abrasive properties of baking soda allow it to serve as a mechanical cleanser on the teeth and gums, to neutralize acid in the mouth, and also to act as an antiseptic to help prevent infections. The baking soda, also adjusts the pH of the tooth whitening composition within a physiologically acceptable range of between about 5.5 and 8.0. A pH of 5.5 is known to be beneficial for remineralization of tooth enamel when used with fluoride.

In some embodiments, the tooth whitening compositions disclosed herein may comprise peppermint oil Peppermint oil is widely used as a topical cooling agent. The essential oil present in peppermint is also known to be effective in killing what are called anaerobic bacteria, which tend to live in low-oxygen environment. It also acts as an alkaline that counters effects of the acidic environment in the oral cavity believed to be responsible for gum related issues. Compositions according to the present disclosure may comprise the following ranges of materials by weight percentage: Papain from 5 percent to 14 percent; Malic acid from 2 to 5 percent; Bromelain from 4 to 11%; Xylitol from 10 to 14 percent; Glycerin from 20 to 24 percent; Peppermint oil from 0.5 to 1.5 percent; Sodium bicarbonate from 2 to 6 percent; and the remainder by weight being water.

An example embodiment of a composition and a manufacturing method for dental whitening wipes comprises the following:

Ina first step of preparing a whitening composition in the form of a gel, xylitol is poured in a mixer along with peppermint oil and mixed together until a homogenous gel is obtained.

Next, glycerin is added to the gel and is mixed together under a vacuum of 28 in. Hg for 60-90 minutes until it is completely dispersed. Mixing may be performed at room temperature, and a water jacket may be provided on the outside surface of the mixer to remove heat generated by the mixing process. The vacuum is then turned off.

Sodium fluoride in powder form (if desired) is then added slowly onto a mixing vessel containing the above described vacuum-blended mixture.

Next, the enzymes papain, malic acid, and bromelain are added into a suitable stainless steel container, and stirred for at least 60 minutes, then are melted at 50-60° C. to ensure a homogeneous clear emulsion is obtained. The emulsion is then allowed to cool until reaching about 40° C.

Next, baking soda (sodium bicarbonate) is added to the above mixture to adjust the pH. The mixture may be heated such as by using a water bath to enable the baking soda dissolve more easily.

The whitening composition according to various embodiments described above may be placed in, that is to moisten an absorbent medium for application to teeth surfaces. In some embodiments, non-woven cloth-like texture material, e.g., cotton fiber or synthetic fiber, may be folded into convenient size folded wipes, for example, 32 mm+30 mm and 2 ply. The folded wipes may be pre-moistened with the whitening composition made as explained above. The folded wipes may be individually wrapped in a layered paper/aluminum bag or packet of convenient size, e.g., 50 mm×50 mm (the outer material) that is easily torn by hand to open the package and remove the wipe.

The wipes may be applied to the user's teeth for 20 seconds or less to clean the surface of the teeth, and may then be used on an as-needed basis as a safe way to whiten teeth. Additional chemicals that may be used in the wipes prior to packaging include an emulsifier to enhance solubility in water-based ingredients, and food additives, as described.

Having explained an example embodiment of making a dental whitening composition, an explanation of various components used in the composition will be provided below.

Papain, also known as papaya proteinase, is an enzyme present in the leaves, roots and fruit of the papaya plant. Papain is usually produced as a crude, dried material by collecting the latex from the fruit of the papaya tree. This latex is then further dried. This purification consists of the solubilization and extraction of the active papain enzyme as powder or as liquid. Papain has wide ranging commercial applications in the cosmetic, detergents, food and pharmaceutical industries. Some studies have also described Papain as having antibacterial action. Papain is also the main ingredient of Papacarie, a gel used to treat tooth decay and is reported to have healing properties that reduce inflammation, and is gentle to dental enamel because it is non acidic.

Malic acid is an organic compound found, for example, in strawberries, blackberries, blueberries, apricots and peaches. Malic acid occurs naturally in many fruits and vegetables, and is generated in fruit metabolism. The process of malolactic fermentation converts malic acid to much milder lactic acid. The malic acid is clear and pure in rhubarb, which is said to have whitening properties, which may help whiten stained teeth. Malic acid can also increase saliva production in people with dry mouth. Saliva protects against tooth decay, a common cause of discoloration, by washing away food and debris.

Bromelain is an enzyme extract derived from the stems of pineapples. The name bromelain refers to either of two protease enzymes extracted from the plants of the family bromeliaceae, or it may refer to a combination of those enzymes along with other compounds produced in an extract. Bromelain extract is a mixture of protein-digesting (proteolytic) enzymes. When used for dental whitening, bromelain has shown to degrade stains on the tooth enamel surface allowing light to reflect off of the enamel surface giving the appearance of whiter teeth visible to the naked eye. During the process of stain whitening using bromelain, the structure of the tooth is not altered itself leaving the fibroblasts untouched.

Xylitol is a natural sweetener that originates from plums, pumpkins, certain other fruits and vegetables. It is a colorless or white crystalline solid that is soluble in water and is classified as a sugar alcohol. Xylitol has negligible effects on blood sugar because it is metabolized independently of insulin. Replacing sugar with xylitol in food products is found to promote better dental health. It is approved in the United States as a food additive. Research has identified oral bacteria as the main pathobiological etiology for dental caries. Xylitol has properties that affect dental caries. While other sugars break down into food for bacteria that spread in the mouth and cause plaque, caries, and gum disease, xylitol is indigestible by common oral bacteria. As a result, the presence of harmful bacteria in the mouth is greatly reduced. Because harmful bacteria produce acids that dissolve or degrade tooth enamel, fewer of such bacteria results in a more balanced pH in the mouth and healthier, stronger teeth. It has been shown that the use of xylitol dental products may in some circumstances restore tooth enamel by producing more alkaline saliva that enables re-mineralization of the teeth.

Glycerin is a simple polyol compound. It is a colorless, odorless, viscous liquid that is sweet-tasting and non-toxic. A glycerol “backbone” is found in lipids known as glycerides. It is also known to have antimicrobial and antiviral properties, which may reduce bacterial activity by reducing the available water activity, which in turn can protect against tooth decay. Glycerin is widely used as a sweetener in the food industry and as a humectant in pharmaceutical formulations with properties to prevent dry mouth. Glycerin is another ingredient in teeth whitening products to expedite stripping of moisture in the tooth enamel. This moisture stripping allows for the whitening product to reach the stains below the surface of the tooth. Furthermore, glycerin is in teeth whitening products to prolong their shelf-life.

Sodium bicarbonate, commonly known as baking soda, has a slightly salty, alkaline taste and also known as baking soda. Because baking soda is salty and alkaline, it can be effective at lightening extrinsic stains caused by acidic foods and drinks such as coffee, red wine, and tea. Baking soda is also effective at killing bacteria in the mouth and can even help freshen breath. Baking soda also serves as an abrasive, making it beneficial for removing plaque and stain build-up. The anti-caries and abrasive properties of baking soda allows it to serve as a mechanical cleanser on the teeth and gums, to neutralize acid in the mouth, and also to act as an antiseptic to help prevent infections. It is used as a buffering agent and can help reduce acid levels in the mouth as this is an alkaline material. It may also counteract the negative impacts with a low pH level because of its ability to quickly dissolve and can enter the smallest cracks in mouth in order to fight bacteria and pH hardness of materials.

Sodium fluoride (NaF) is an inorganic compound used in trace amounts in the fluoridation of drinking water, in dentrifice (toothpaste), and is also used in pesticides. It is a colorless or white solid that is readily soluble in water. It is a common source of fluoride in the production of pharmaceuticals and is used to prevent dental cavities. Sodium fluoride makes teeth more resistant to decay and bacteria that cause cavities. It is used to prevent cavities, reduce tooth sensitivity, whitens teeth, and provide corrective action from acid damage and reduce complications related to dry mouth. The fluoride enhances the strength of teeth by the formation of fluorapatite, a naturally occurring component of tooth enamel. Sodium fluoride when applied to teeth can prevent cavities. Fluoride is a mineral that works by making the teeth stronger and more resistant to decay caused by acid and bacteria.

Charcoal used as an abrasive agent helps keep tooth enamel strong, which helps to protect the teeth against cavities and decay and may improve halitosis (bad breath). Activated carbon, also called activated charcoal, is usually derived from waste products such as coconut husks. It is processed to have small, low-volume pores and to be porous enough to absorb certain toxins by increasing the surface area available for adsorption.

The whitening composition according to various embodiments described above may be placed in, that is to moisten an absorbent medium for application to teeth surfaces. In some embodiments, non-woven cloth-like texture material, e.g., cotton fiber or synthetic fiber, may be folded into convenient size folded wipes, for example, 32 mm×30 mm and 2 ply. The folded wipes may be pre-moistened with the whitening composition made as explained above. The folded wipes may be individually wrapped in a layered paper/aluminum bag or packet of convenient size, e.g., 50 mm×50 mm (the outer material) that is easily torn by hand to open the package and remove the wipe.

The wipes may be applied to the user's teeth for 20 seconds or less to clean the surface of the teeth, and may then be used on an as-needed basis as a safe way to prevent the discoloration of teeth. Additional chemicals that may be used in the wipes prior to packaging include an emulsifier to enhance solubility in water-based ingredients, and food additives, as described.

Teeth whitening may be performed by removal of stains through certain proteins found in fruit enzymes that speeds up a chemical reaction and some abrasion to lighten the tooth color. In other words, the frequent and short-time use of whitening composition treatments according to the present disclosure may obtain the same desired whiteness, it will also protect from enamel erosion and damage to teeth as compared with using peroxide compositions known in the art for dental whitening.

In light of the principles and example embodiments described and illustrated herein, it will be recognized that the example embodiments can be modified in arrangement and detail without departing from such principles. The foregoing discussion has provided specific embodiments, but other configurations are also contemplated. In particular, even though expressions such as in “an embodiment,” or the like are used herein, these phrases are meant to generally reference embodiment possibilities, and are not intended to limit the disclosure to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise. Although only a few examples have been described in detail above, those skilled in the art will readily appreciate that many modifications are possible within the scope of the described examples. Accordingly, all such modifications are intended to be included within the scope of this disclosure as defined in the following claims.

What is claimed is:

1. A dental whitening composition, comprising:
an effective amount of at least one fruit enzyme;
an effective amount of at least one fruit sweetener;
an effective amount of abrasive material; and
wherein the at least one fruit enzyme, the at least one fruit sweetener are in an aqueous suspension with the abrasive material.

2. The composition of claim 1 wherein the at least one fruit enzyme comprises one or more of papain, malic acid, and bromelain.

3. The composition of claim 1 wherein the abrasive materials comprises charcoal and/or sodium bicarbonate.

4. The composition of claim 1 further comprising an effective amount of peppermint oil.

5. The composition of claim 1 further comprising an effective amount of sodium fluoride.

6. The composition of claim 1 further comprising an effective amount of glycerin.

7. The composition of claim 1 wherein:
the at least one fruit enzyme comprises papain in a range of 5 to 14 percent by weight, malic acid in a range of 2 to 5 percent by weight, and bromelain in a range of 4 to 11 percent by weight;
the at least one fruit sweetener comprises xylitol in a range of 10 to 24 percent by weight, the abrasive material comprises sodium bicarbonate in a range of 2 to 6 percent;
a thickening material comprising glycerin is in a range of 20 to 24 percent by weight; and
a remainder of the mixture comprises water.

8. The composition of claim 7 further comprising peppermint oil in a range of 0.5 to 1.5 percent by weight, and wherein the water is reduced in amount correspondingly to an amount of the peppermint oil.

9. A dental whitening wipe, comprising:
an effective amount of at least one fruit enzyme;
an effective amount of fruit sweetener;
an effective amount of abrasive material;
wherein the at least one fruit enzyme, the at least one fruit sweetener, and the abrasive material are applied to an absorbent non-woven fabric-like material for temporary application to teeth.

10. The wipe of claim 9 wherein the at least one fruit enzyme comprises one or more of papain, malic acid or bromelain.

11. The wipe of claim 9 wherein the abrasive materials comprises charcoal and/or sodium bicarbonate.

12. The wipe of claim 9 further comprising an effective amount of peppermint oil.

13. The wipe of claim 9 further comprising an effective amount of natural sweetener xylitol.

14. The wipe of claim 9 further comprising an effective amount of glycerin.

15. The wipe of claim 9 wherein:
the at least one fruit enzyme comprises papain in a range of 5 to 14 percent by weight, malic acid in a range of 2 to 5 percent by weight, and bromelain in a range of 2 to 3 percent by weight;
the abrasive material comprises sodium bicarbonate in a range of 2 to 3 percent;
the at least one fruit sweetener comprises xylitol in a range of 10 to 24 percent by weight; and
a remainder of the mixture comprises water.

16. The wipe of claim 15 further comprising peppermint oil in a range of 2 to 3 percent by weight, and wherein the water is reduced in amount correspondingly to an amount of the peppermint oil.

17. The wipe of claim 15 further comprising glycerin in a range of 20 to 24 percent, and wherein the water is reduced in amount corresponding to an amount of the glycerin.
